(19) United States
(12) Patent Application Publication
(10) Pub. No.: US 2013/0018215 A1
(43) Pub. Date: Jan. 17, 2013

(54) ESOPHAGEAL STENT AND METHODS FOR USE OF SAME

(71) Applicants: Rich Snider, Dallas, TX (US); James S. Barthel, Tampa, FL (US)

(72) Inventors: Rich Snider, Dallas, TX (US); James S. Barthel, Tampa, FL (US)

(73) Assignee: Merit Medical Systems, Inc., South Jordan, UT (US)

(21) Appl. No.: 13/352,926

(22) Filed: Jan. 18, 2012

Related U.S. Application Data
(60) Provisional application No. 61/433,818, filed on Jan. 18, 2011.

(51) Int. Cl. A61F 2/04; A61N 5/10; A61F 2/84

(52) U.S. Cl. 600/1; 623/23.7

(57) ABSTRACT

Disclosed herein are various embodiments of esophageal stents and related methods for the treatment of esophageal dysphagia. Esophageal stents, according to the present disclosure, may have a diameter between 8 mm and 16 mm. Further, esophageal stents according to the present disclosure may have a length between 40 and 150 mm. A method, according to the present disclosure, may include the steps of placing an esophageal stent across an esophageal stricture, treating an underlying cause of the esophageal stricture, and retrieving the esophageal stent following migration of the esophageal stent. The esophageal stents of the present disclosure may be configured to avoid radial and other forces that cause undue strain and/or stretching of the esophageal tissue.

### RELATED APPLICATIONS

This application claims the benefit under 35 U.S.C. §119(e) of U.S. Provisional Patent Application No. 61/433,818, filed Jan. 18, 2011, and titled “ESOPHAGEAL STENT AND METHODS FOR USE OF SAME,” which is hereby incorporated by reference herein in its entirety.

### TECHNICAL FIELD

The present disclosure relates generally to devices configured to be implanted within a body lumen. More particularly, the present disclosure relates to stents or similar prosthetic devices which, in certain embodiments, are configured to be disposed within the esophagus.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A is a plan view of one embodiment of an esophageal stent.

FIG. 1B is a plan view of a portion of the esophageal stent of FIG. 1A.

FIG. 1C is an end view of the esophageal stent of FIG. 1A.

FIG. 2A is a plan view of one embodiment of an esophageal stent including a cover.

FIG. 2B is an enlarged view of a portion of the esophageal stent of FIG. 2A.

FIG. 3 is a plan view of a portion of one embodiment of an esophageal stent including a suture.

### DETAILED DESCRIPTION

Disclosed herein are various embodiments of esophageal stents and methods for use of the same. Self-expanding metal stents (SEMS) and self-expanding plastic stents (SEPS) may be utilized for palliation of dysphagia. Dysphagia may be caused by a variety of medical conditions, including malignant growth in or around the esophagus. Disclosed herein are various embodiments and methods of use of both SEMS and SEPS in patients with esophageal dysphagia.

Malignant dysphagia is one form of dysphagia that may be treated using the systems and methods disclosed herein. Malignant dysphagia is defined as the inability to swallow normally as a consequence of cancer. This condition may result from intrinsic tumor involvement or extrinsic compression of the esophagus. Malignant dysphagia also may be caused or complicated by the development of tracheoesophageal fistula (TEF). Malignant dysphagia is commonly caused by esophageal and lung cancers and TEF may be formed by malignant invasion from either type of primary tumor or may result from loss of tumor tissue following treatment.

Tumors causing extrinsic compression of the esophagus also may be responsible for malignant dysphagia. Malignant dysphagia, with or without TEF, may be caused by metastatic breast cancer, liposarcoma, lymphomas, lung cancers, and other lesions. Although there is some evidence that better relief from dysphagia can be obtained when stents are utilized for intrinsic obstruction than for extrinsic obstruction, stents may be used to treat malignant dysphagia in cases involving both intrinsic obstruction and extrinsic obstruction of the esophagus.

Esophageal stents may be placed using a combination of endoscopy and fluoroscopy and/or under direct endoscopic visualization alone without the aid of fluoroscopy.

An esophageal stent may be used to minimize symptoms associated with esophageal dysphagia and to promote oral intake of food, water, and medications; seal fistulas or anastomotic leaks following surgical interventions; and improve a patient's quality of life. A standardized system for grading dysphagia, based on a numeric 0 to 4 scale, is available to quantify swallowing status and has been widely utilized (Table 1). Esophageal stents may assist in improving a patient's dysphagia score and treatment.

Dysphagia Scoring System and Related Symptoms

| | |
|---|---|
| 0 | No dysphagia |
| 1 | Dysphagia to normal solids |
| 2 | Dysphagia to soft solids |
| 3 | Dysphagia to solids and liquids |
| 4 | Inability to swallow saliva |

Stent diameter may affect long-term results of stent placement, both because of differences in radial expansile force and stent circumference at full expansion. For example, larger diameter stents may have an increased risk of hemorrhage, perforation, or fistula when compared with smaller diameter stents; however, smaller diameter stents are more likely to be complicated by stent migration, tissue overgrowth, and food bolus impaction, requiring more frequent interventions. Further, stent length may also affect long-term results of stent placement. Longer stents may be less likely to migrate within a patient's gastrointestinal tract.

Stent diameter may also affect long-term results of treatments to ameliorate or resolve an underlying cause of the esophageal dysphagia, such as radiation therapy and/or chemotherapy to treat a tumor. Tissue that is in a strained or stretched state may be more susceptible to damage—possibly irreversible damage—when exposed to treatments, such as radiation therapy and/or chemo therapy. Strained or stretched tissue may tend to scar more easily. Unduly strained/stretched tissue, in particular, may damage and scar more easily than tissue in a natural or substantially unstrained state. In esophageal tissue, scarring and other tissue damage can itself cause dysphagia.

SEMS may be made of a variety of metals and alloys, including nitinol (nickel-titanium alloy), stainless steel, or cobalt-chromium-nickel alloy and may be coated with silicon or polyurethane. Uncovered SEMS may expand into a tumor, with stent incorporation into the submucosal space of the esophageal wall developing in as few as 18 days. Covered stents may pose a lower risk of obstruction by ingrowth of tumor, but covered stents may also have an associated increased risk of migration.

SEPS may also be utilized as an alternative to SEMS. SEPS may be less expensive than SEMS. SEPS may also be well suited for sealing anastomotic leaks following esophageal surgery for both benign and malignant diseases. Drawbacks to SEPS may include a wider and more cumbersome delivery system. SEPS may also have a higher incidence of stent migration, which may be due, in part, to the fully coated nature of the devices as well as the applications for which they are utilized.

The choice of stent and the physical properties of the stent (e.g., the length and diameter of the stent) should be highly individualized and based on patient characteristics, tumor type, and location, as well as the patient's overall prognosis and treatment plan. Through relief of the symptoms of dysphagia, esophageal stents provide a relatively low-risk option for improving quality of life and nutrition in patients with malignant dysphagia.

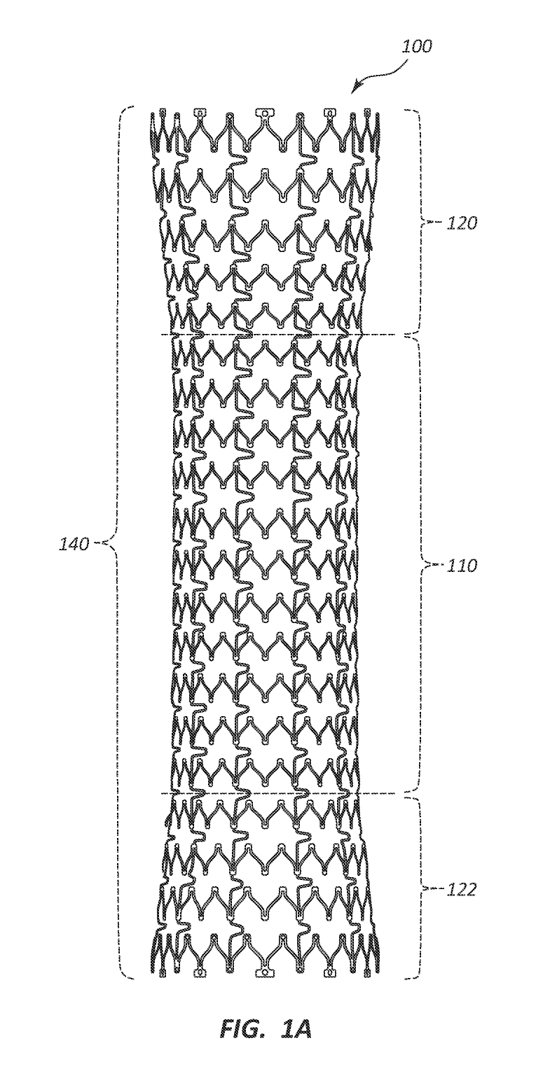

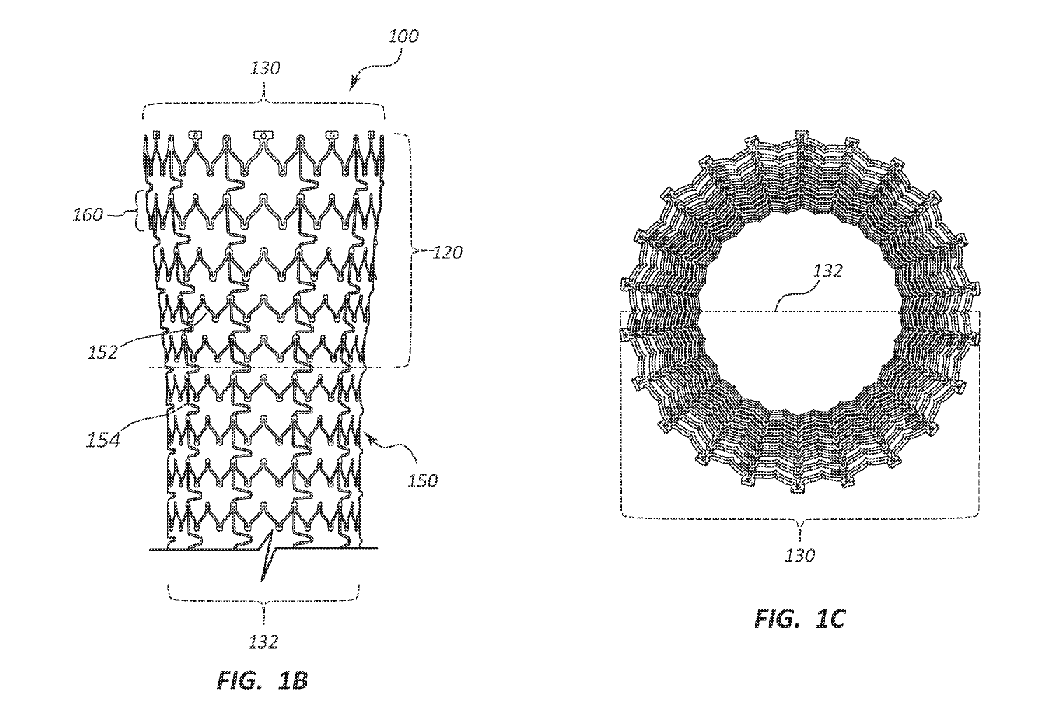

With reference to the accompanying figures, FIG. 1A illustrates a perspective view of one embodiment of an esophageal stent 100. The illustrated esophageal stent 100 has a length 140. The length of a stent for use in a particular case may be determined by the condition of the patient and by the likelihood of undesired stent migration. Embodiments having a longer length may be less prone to undesired stent migration. According to various embodiments, length 140 of esophageal stent 100 may be between 40 and 150 mm. According to other embodiments, length 140 of esophageal stent 100 may be between 70 and 120 mm. In still other embodiments, length 140 may be between 80 and 120 mm. Finally, according to certain embodiments, length 140 may be between 90 and 120 mm. Stent 100 includes flared regions 120 and 122, which are disposed at opposite ends of esophageal stent 100. A central region 110 is disposed between flared regions 120 and 122.

As illustrated in FIG. 1B, flared region 120 may have an opening diameter 130. The diameter of flared region 120 may decrease along its length, and central region 110 may have a narrower diameter 132. Diameter 132 of central region 110 may be approximately consistent along the length of central region 110. The larger diameter 130 of flared region 120 may help to direct food or liquid ingested by a patient into central region 110 of esophageal stent 100, and may further help to secure esophageal stent 100 in a patient's esophagus, thus helping to prevent undesired stent migration. Diameter 132 of central region 110 may also influence the occurrence of undesired stent migration. According to various embodiments, diameter 132 may be between 8 and 16 mm. According to other embodiments, diameter 132 may be between 10 and 16 mm. Finally, according to certain embodiments, diameter 132 may be between 10 and 14 mm.

FIG. 1C illustrates an end view of stent 100. Further, FIG. 1C illustrates the larger diameter 130 of flared region 120 and the narrower diameter 132 of central region 110.

Stent 100 is shown in FIGS. 1A-1C as having interstice geometry. As illustrated in FIG. 1B, stent 100 includes a stent scaffolding 150 formed by a plurality of struts. The struts include a plurality of interconnected legs 152 and connectors 154. Esophageal stent 100 includes a series of legs 152 arranged circumferentially about the stent rows or annular segments 160. The annular segments 160 are arranged along the longitudinal axis of esophageal stent 100, while a plurality of connectors 154 are arranged parallel to the longitudinal axis of esophageal stent 100 to connect the annular segments 160 together. Esophageal stent 100 may be formed of a memory metal that facilitates flexibility of esophageal stent 100, such that stent 100 may be deformed and return to its original shape. As such, legs 152 and connectors 154 of stent 100 may be formed from a composite material, such as Ni, C, Co, Cu, Cr, H, Fe, Nb, O, Ti and combinations thereof (e.g., nitinol). The composite material is generally formed into a compressed tube from which the stent is etched and then formed on a suitable shaping device to give the stent the desired external geometry.

Stents in accordance with the present disclosure may include any number of combinations of interstice geometry, which may be realized by changing angles, strut lengths, strut thicknesses, annular segment lengths, and annular segment thicknesses during the etching and forming stages of stent engineering or during post formation processing and polishing steps. Moreover, by modifying the geometry of the plurality of connectors 154, additional functionality may be achieved.

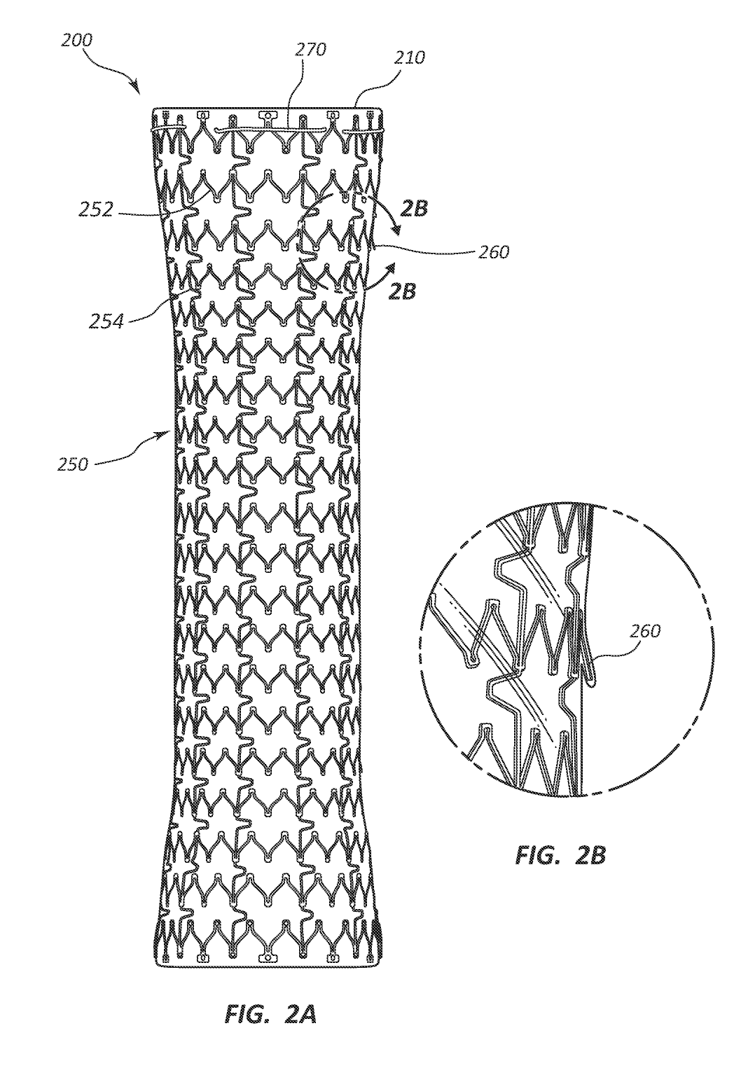

FIG. 2A illustrates an embodiment of an esophageal stent 200 including a cover 210. Cover 210 may be formed of a polymer, such as polyurethanes (e.g., polycarbonate urethane, or Chronoflex® manufactured by Cardiotech International). Cover 210 may be applied between a plurality of legs 252 and connectors 254, and may graft each of the legs 242 and connectors 254 into a unitary stent structure (e.g., a continuous surface). According to certain embodiments, cover 210 may be formed of a material that does not inhibit flexing or radial expansion of stent 200; however, according to other embodiments, cover 210 may be designed to control one or more physical properties (e.g., flexing and radial expansion) of stent 200.

According to various embodiments, legs 252 and connectors 254 may be raised above the surface of cover 210. Thus, cover 210 may be applied to the interior of stent 200 such that the stent scaffolding 250 formed by the plurality of legs 252 and connectors 254 is raised above the surface of cover 210 (e.g., 1 A to 10⁶ Å). According to other embodiments, cover 210 may be applied to the exterior of the stent scaffolding 250, or may be applied on both sides of the stent scaffolding 250.

FIG. 2B illustrates a scale 260 extending outwardly from the outer surface of the stent 200. Scale 260 may be formed of one or more legs 252 that protrude in a manner that inhibits and/or prevents undesired migration of stent 200. For example, scale 260 may inhibit migration of stent 200 in a downward direction (e.g., in the direction of the patient's stomach). Scale 260 may extend from the outer surface in any number of orientations. Scale 260 may define an acute angle between the protruding leg(s) 252 and the outer surface of stent 200, although various angles may be incorporated in additional embodiments. One or more scales 260 may extend from the outer surface of stent 200 in any desired location. For example, in the embodiment illustrated in FIG. 2A, the scales are located in the third or fourth row or annular segment of legs 252.

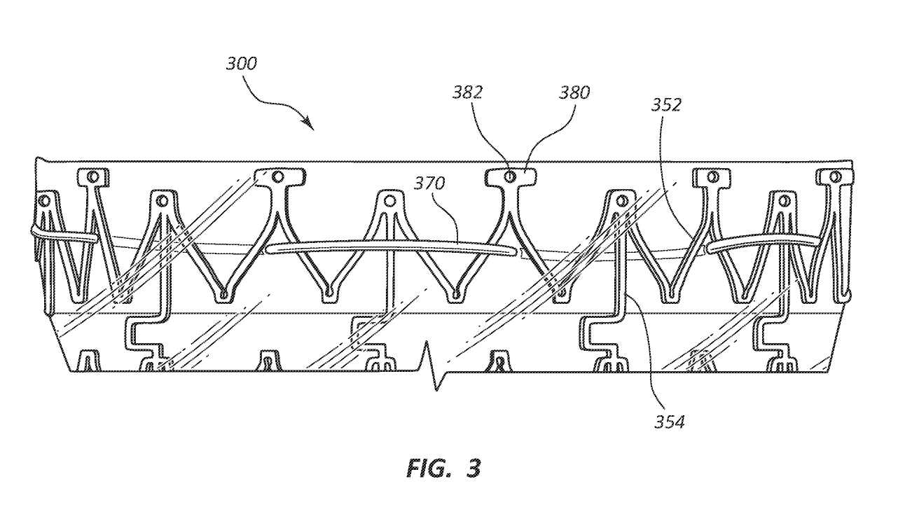

A suture 270 may be disposed around a circumference of the stent 200. The suture may be formed of any suitable suture material, as known to those skilled in the art, such as polypropylene. The term “suture,” as used herein, includes any suitable thread or wire capable of enduring the forces applied during an initial placement, repositioning, or removal of stent 200. FIG. 3 illustrates in greater detail the function of suture, according to various embodiments.

FIG. 3 is a plan view of a portion of one embodiment of an esophageal stent 300, including a suture 370. FIG. 3 illustrates suture 370 intertwined about a plurality of legs 352 and connectors 354 of stent 300. Suture 370 may be disposed proximate to, and circumferentially about, at least one opening of stent 300. Suture 370 may be intertwined about a plurality of legs 352 and connectors 354, such that a force applied through the suture 370 transfers force to the stent. FIG. 3 illustrates that stent 300 may include ends 380 having apertures 382 defined therein through which the suture 370 may be threaded in order to purse string the stent.

Various embodiments of esophageal stents disclosed herein may be utilized in connection with methods for the treatment of esophageal dysphagia. According to one embodiment, the method may include the steps of placing an esophageal stent across an esophageal stricture, treating an underlying cause of the esophageal stricture, and retrieving the esophageal stent following migration of the esophageal stent after treatment. A period of time may be given to allow for migration of the stent. Migration of the stent may be used as an indicator of effective treatment of the underlying cause of the stricture. In other words, in some instances, migration of the stent may be intended.

The esophageal stent that is used in connection with the disclosed methods for the treatment of esophageal dysphagia may be sized and configured to open a target esophageal stricture while avoiding undue stress on the mucosa. In other words, the esophageal stent may be configured to achieve radial forces that open an esophageal stricture and maintain palliation, while maintaining the surrounding esophageal tissue in a substantially unstrained state (e.g., an unstretched state) or without otherwise unduly stretching the esophageal tissue.

Subsequent treatment of the underlying cause of the esophageal stricture may include radiation therapy and/or chemotherapy to treat, for example, a tumor causing the stricture. Tissue that is in a strained or stretched state may be more susceptible to damage and/or scarring when exposed to treatments, such as radiation therapy and/or chemo therapy. By using an esophageal stent that is sized and configured (stent profile and stent design to achieve radial forces which maintain palliation without stressing the mucosa) to maintain the esophageal tissue in a neutral and/or unstrained state, unnecessary tissue damage that may result from the subsequent treatment can be limited or avoided.

In some cases, the treatment of the underlying cause of the esophageal stricture is effective and reduces or even eliminates the cause of the stricture. In such cases, the esophagus may return to its state prior to the stricture and prior to the dysphagia. Because the stent used is configured to open the stricture while maintaining the surrounding esophageal tissue in an unstrained state, resolution of the underlying cause of the stricture may reduce or eliminate the stricture and leave the stent applying relatively little outward force to a wall of the esophagus. The stent may be left relatively loosely disposed in the esophagus. This may result in migration of the stent. The migration of the stent may be allowed, anticipated, and/or intended.

The migration of the stent may be distally, deeper into the patient's gastrointestinal tract. In some cases, the stent may migrate distally to interfere with the lower esophageal sphincter (LES). The stent may inhibit proper functioning of the LES, preventing the LES from closing. In other cases, the stent may migrate into the stomach. In still other cases, the stent may migrate into the small intestine. The disclosed methods may include retrieval of the stent following migration of the stent after treatment of the underlying cause of the stricture.

Retrieval of the stent may be accomplished by a medical procedure with the aid of an endoscope and/or fluoroscopy. A stent removal sheath may be inserted down the esophagus and adjacent to the stent to be retrieved, whether in the esophagus or stomach. According to one embodiment, a physician, with the aid of an endoscope and/or fluoroscopy may use a stent removal instrument to engage or otherwise exert a force (e.g., pull or push) on a suture disposed around a circumference of the stent. The force on the suture may transfer to the stent and cause at least a portion of the stent (e.g., an end of the stent) to purse-string and/or collapse. The collapsed portion of the stent may be drawn into the stent removal sheath or pulled directly through the esophagus for safe removal from the body. The stent removal instrument may also be used to draw the stent into the stent removal sheath.

Various embodiments of methods according to the present disclosure may include placing an esophageal stent having a length between 40 and 150 mm. Further, various embodiments of methods according to the present disclosure may include placing an esophageal stent having a diameter between 8 and 16 mm.

Provided below are specific examples of the use of various embodiments of esophageal stents and related methods, according to the present disclosure.

### EXAMPLE 1

Three patients received small caliber esophageal stents, with internal diameters ranging from 12 mm to 16 mm, and having the same structure as the stent shown in FIG. 1A. Endoscopy reports were reviewed and post procedure phone surveys were performed to assess dysphagia scores and degree of chest pain.

Post-stent median dysphagia score improved to 2 from 4 in all three patients. None of the patients had post procedure chest pain. One patient experienced stent migration, which resulted after tumor reduction from chemoradiation. The patient was able to maintain oral nutrition throughout chemoradiation and the stent was successfully removed from the stomach. The other two patients experienced saliva control and were tolerating a semi-solid diet. They declined further treatment and opted for hospice care. With successful multimodality therapy, stent migration may be considered an endpoint of therapy and not a complication of stent placement. Small caliber SEMS placed for palliation of malignant dysphagia in patients whose life expectancy is less than three months may permit adequate saliva control and nutritional intake without major complications.

### EXAMPLE 2

A prospective observational study of 17 patients presenting with severe malignant dysphagia from either esophageal adenocarcinoma or squamous cell cancer was performed. Thirty-one SEMS with internal diameters ranging from 8 mm to 16 mm were placed in seventeen patients with pre-stent residual luminal diameters of <8 mm (n=13) or 8-10 mm (n=4). Seven stents with internal diameters ranging from 14 mm to 16 mm, two stents with internal diameters ranging from 8 mm to 10 mm, and twenty-two specially constructed stents with internal diameters in the range of 12 mm to 16 mm were placed. The twenty-two specially constructed stents had essentially the same structure as the stent 100 shown in FIG. 1A. All stents were placed under direct endoscopic vision without fluoroscopic support. Dysphagia scores before and after stent placement, migration rates (both anticipated migration and unanticipated migration), and complications were identified through review of endoscopy reports, outpatient clinical encounters, and standardized 24-hour post-procedure phone surveys. Anticipated stent migration was defined as events which occurred in patients receiving tumor reduction chemo radiation. Unanticipated stent migration was defined as events which occurred in the absence of treatment.

The post-stent median dysphagia score improved (decreased) from 3 to 2 (P=0.0003). The overall median duration of first stent placement was 64 days, 1 QR 32-110 days. The overall migration rate was 35.5% (11/31). The anticipated migration rate was 60% (9/15) while the unanticipated migration rate was 18.2% (2/11) (P=0.033). All of the migrated stents were retrieved endoscopically without complication. 9.7% (3/31) of patients reported self-limited sore throat or chest pain within 24-hours of stent placement. All three of these patients were managed conservatively without need for narcotic analgesics, hospitalization, or repeat endoscopy. No other complications occurred.

### EXAMPLE 3

Prospective observational study was performed involving patients presenting with severe malignant dysphagia from esophageal adenocarcinoma or squamous cell cancer between Dec. 1, 2008, and Nov. 1, 2010. Thirty-eight SEMS with internal diameters ranging from 8 mm to 16 mm were placed in twenty-three patients with pre-stent luminal diameters of <8 mm (n=18) or 8-10 mm (n=5). Eight stents with internal diameters ranging from 14 mm to 16 mm, two stents with internal diameters 8 mm to 10 mm, and twenty-eight specially constructed stents with internal diameters ranging from 12 mm to 16 mm were placed. The twenty-eight specially constructed stents had essentially the same structure as the stent 100 shown in FIG. 1A. All stents were placed under direct endoscopic vision without fluoroscopic support. Dysphagia scores, migration rates (both anticipated migration and unanticipated migration), and complications were identified through review of endoscopy reports, outpatient clinical encounters, and 24-hour post-procedure phone surveys. Anticipated stent migration was defined as events which occurred in patients receiving tumor reduction chemoradiation. Unanticipated stent migration was defined as events which occurred in the absence of treatment.

The post-stent median dysphagia score improved (decreased) from 3 to 2 (P<0.0001). The overall mean duration of first stent placement was seventy-one days, over a range of seven days to one hundred and thirty-nine days. 10/23 patients have a stent in place currently and are receiving treatment; 8/23 died with stent; 3/23 had stents removed after anticipated migration and are swallowing adequately; 1/23 served as a successful bridge to esophagectomy; 1/23 required elective removal due to chest pain. The overall migration rate was 31.6% (12/38). The anticipated migration rate was 45.5% (10/22); the unanticipated migration rate was 12.5% (2/16) (P−0.040). All 12 of the migrated stents were retrieved endoscopically without complication. 13.2% (5/38) of patients reported self-limited sore throat or chest pain within 24-hours of stent placement. All 3 patients were managed conservatively without need for hospitalization or repeat endoscopy. No other complications occurred.

Various modifications, changes, and variations apparent to those of skill in the art may be made in the arrangement, operation, and details of the disclosed embodiments without departing from the spirit and scope of the disclosure. Thus, it is to be understood that the embodiments described above have been presented by way of example, and not limitation.

Where a range of values is provided, it is understood that each intervening value, to the tenth of the unit of the lower limit unless the context clearly dictates otherwise, between the upper and lower limit of that range and any other stated or intervening value in that stated range is encompassed within the embodiments of the present invention. The upper and lower limits of these smaller ranges which may independently be included in the smaller ranges is also encompassed within the embodiments of the invention, subject to any specifically excluded limit in the stated range. Where the stated range includes one or both of the limits, ranges excluding either or both of those included limits are also included in the embodiments of the invention.

Unless defined otherwise, all technical and scientific terms used herein have the same meaning as commonly understood by one of ordinary skill in the art to which the embodiments of the invention belong. Although any methods and materials similar or equivalent to those described herein can also be used in the practice or testing of the embodiments of the present invention, the preferred methods and materials are now described. All patents and publications mentioned herein, including those cited in the Background of the application, are hereby incorporated by reference to disclose and describe the methods and/or materials in connection with which the publications are cited.

Other embodiments of the invention are possible. Although the description above contains much specificity, these should not be construed as limiting the scope of the invention, but as merely providing illustrations of some of the presently preferred embodiments of this invention. It is also contemplated that various combinations or sub-combinations of the specific features and aspects of the embodiments may be made and still fall within the scope of the invention. It should be understood that various features and aspects of the disclosed embodiments can be combined with or substituted for one another in order to form varying modes of the disclosed embodiments of the present invention. Thus, it is intended that the scope of at least some of the present invention herein disclosed should not be limited by the particular disclosed embodiments described above.

It will be obvious to those having skill in the art that many changes may be made to the details of the above-described embodiments without departing from the underlying principles of the invention. The scope of the present invention should, therefore, be determined only by the following claims.

1. An esophageal stent, comprising:
a scaffolding comprising a plurality of struts formed from a tube of memory material and having a proximal end and a distal end;
a cover applied to the scaffolding of struts to define an interior region within the scaffolding;
a first flared region and a second flared region, each formed by the scaffolding and disposed at the proximal end and the distal end, respectively, the first flared region and the second flared region having a diameter that varies along their respective lengths; and
a central region disposed between the first flared region and the second flared region, the central region having an approximately constant diameter,
wherein the central region has a diameter between 8 mm and 16 mm.

2. The esophageal stent of claim 1, wherein the central region of the esophageal stent has a diameter between 10 and 16 mm.

3. The esophageal stent of claim 1, wherein the central region of the esophageal stent has a diameter between 10 and 14 mm.

4. The esophageal stent of claim 1, wherein the esophageal stent has a length between 40 and 150 mm.

5. The esophageal stent of claim 1, wherein the esophageal stent has a length between 70 and 120 mm.

6. The esophageal stent of claim 1, wherein the esophageal stent has a length between 80 and 120 mm.

7. The esophageal stent of claim 1, wherein the esophageal stent has a length between 90 and 120 mm.

8. A method of treating esophageal dysphagia, comprising:
placing an esophageal stent across an esophageal stricture in an esophagus, the esophageal stent comprising a first flared region, a second flared region, and a central region disposed between the first flared region and the second flared region, the central region having an approximately constant diameter between 8mm and 16 mm;
treating an underlying cause of the esophageal stricture; and
retrieving the esophageal stent following migration of the esophageal stent.

9. The method of claim 8, further comprising:
allowing the esophageal stent to migrate after the esophageal stricture is opened by treating the underlying cause of the stricture.

10. The method of claim 8, wherein the stent is configured to open the esophageal stricture while maintaining surrounding esophageal tissue in a neutral unstrained state.

11. The method of claim 10, wherein treating comprises radiation treatment of a tumor causing the esophageal stricture.

12. The method of claim 10, wherein treating comprises chemotherapy treatment of a tumor causing the esophageal stricture.

13. The method of claim 8, wherein the esophageal stent further comprises a cover applied to the scaffolding of struts to define an interior region within the scaffolding and a continuous outer surface.

14. The method of claim 8, wherein retrieving the esophageal stent comprises a medical procedure performed with aid of an endoscope.

15. The method of claim 8, wherein retrieving the esophageal stent comprises a medical procedure performed with aid of fluoroscopy.

16. The method of claim 8, wherein retrieving the esophageal stent comprises using an instrument inserted into the esophagus to engage a suture disposed around a circumference of the esophageal stent and exert a force on the suture to collapse a portion of the esophageal stent.

17. The method of claim 16, wherein retrieving the esophageal stent further comprises using the instrument to draw the collapsed portion of the esophageal stent into a stent removal sheath for removal from the esophagus.

18. The method of claim 8, wherein the esophageal stent has a diameter between 10 and 16 mm.

19. The method of claim 8, wherein the esophageal stent has a diameter between 10 and 14 mm.

20. The method of claim 8, wherein the esophageal stent comprises a scaffolding formed of a plurality of struts formed from a tube of memory material and having a proximal end and a distal end.

21. The method of claim 20, wherein the first flared region and the second flared region are each formed by the scaffolding and disposed at the proximal end and the distal end, respectively, the first flared region and the second flared region having a diameter that varies along their respective lengths.

22. The method of claim 8, wherein the esophageal stent has a length between 80 and 120 mm.

23. The method of claim 8, wherein the esophageal stent has a length between 90 and 120 mm.

24. A method of treating esophageal dysphagia, comprising:
placing an esophageal stent across an esophageal stricture in an esophagus, the esophageal stent comprising:
a scaffolding comprising a plurality of struts formed from a tube of memory material and having a proximal end and a distal end;
a first flared region and a second flared region, each formed by the scaffolding and disposed at the proximal end and the distal end, respectively, the first flared region and the second flared region having a diameter that varies along their respective lengths;
a central region disposed between the first flared region and the second flared region, the central region having an approximately constant diameter between 8 mm and 16 mm;
treating an underlying cause of the esophageal stricture using a chemo radiation tumor reduction treatment; and
retrieving the esophageal stent following migration of the esophageal stent.
